(19) United States
(12) Patent Application Publication
(10) Pub. No.: US 2007/0122374 A1
(43) Pub. Date: May 31, 2007

(54) Waterless urinal formulations

(76) Inventor: Bhupendra Trivedi, Lombard, IL (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/287,903

(22) Filed: Nov. 28, 2005

(51) Int. Cl. A61L 9/00

(52) U.S. Cl. 424/76.2

(57) ABSTRACT

The present invention relates to A composition for use in urinals, comprising: (a) one or more aliphatic alcohols, the aliphatic alcohols comprising 11 carbon atoms or less; (b) one or more esters selected from molecules of formula R¹—C(O)—O—R² and triglycerides of formula CH₂COOR⁶—CHCOOR⁷—CH₂—COOR⁸, R¹ comprising 6 to 24 carbon atoms, R² comprising 1 to 8 carbon atoms, R⁶ comprising 1 to 24 carbon atoms, R⁷ comprising 1 to 24 carbon atoms and R⁸ comprising 1 to 24 carbon atoms; (c) one or more molecules of formula R³—(O)—C(O)—R⁴—C(O)—(O)—R⁵, wherein R³, R⁴ and R⁵ are independently chosen from an alkyl or alkenyl moiety with 1 to 15 carbon atoms; (d) one or more aliphatic hydrocarbons, the aliphatic hydrocarbons comprising 6 to 23 carbon atoms; (e) optionally one or more vegetable oil; (f) optionally one or more unsaturated hydrocarbons, the unsaturated hydrocarbons comprising 6 to 24 carbon atoms; and (g) optionally one or more alicyclic hydrocarbons, the alyclic hydrocarbon comprising 6 to 23 carbon atoms.

### INTRODUCTION

Waterless urinals are becoming more widely used because of costs savings attributed to conservation of water. These waterless urinals are not flushed with water each time a person uses the urinal. As the waterless urinal is repeatedly used, urine is collected in a compartment of the urinal. An oily sealing liquid that is immiscible with the urine and is lighter than the urine covers the collected urine. This oily sealing liquid floats on the surface of the urine, serving as a barrier that prevents odors from the urinal escaping to the environment. In order to fully prevent or minimize the escaping of such odors, the sealing liquid must be viscous enough to preclude the release of the volatile components or derivatives of the waste collected in the urinal. However, the minimization of odors also requires that the sealing liquid be permeable enough to allow incoming urine to quickly pass through, and seal itself as soon as possible. The sealing liquid thus has to have a right combination of density and viscosity, and allow a relatively rapid passage of urine to provide a rapid seal.

### SUMMARY

These and other features of the present teachings are set forth herein. In some embodiments, the present invention teaches a composition for use in urinals, comprising: (a) one or more aliphatic alcohols, the aliphatic alcohols comprising 11 carbon atoms or less; (b) one or more esters selected from molecules of formula R¹—C(O)—O—R² and triglycerides of formula CH₂COOR⁶—CHCOOR⁷—CH₂—COOR⁸, R¹ comprising 6 to 24 carbon atoms, R² comprising 1 to 8 carbon atoms, R⁶ comprising 1 to 24 carbon atoms, R⁷ comprising 1 to 24 carbon atoms and R⁸ comprising 1 to 24 carbon atoms; (c) one or more molecules of formula R³—(O)—C(O)—R⁴—C(O)—(O)—R⁵, wherein R³, R⁴ and R⁵ are independently chosen from an alkyl or alkenyl moiety with 1 to 15 carbon atoms; (d) one or more aliphatic hydrocarbons, the aliphatic hydrocarbons comprising 6 to 23 carbon atoms; (e) optionally one or more vegetable oils; (f) optionally one or more unsaturated hydrocarbons, the unsaturated hydrocarbons comprising 6 to 24 carbon atoms; and (g) optionally one or more alicyclic hydrocarbons, the alyclic hydrocarbon comprising 6 to 23 carbon atoms.

In a second set of embodiments, the present invention teaches a composition for use in urinals, comprising 2-ethylhexanol, dioctyl maleate, n-butyl-propoxylate, one or more aliphatic hydrocarbons comprising 13 to 23 carbon atoms, soybean oil and triclosan.

In a third set of embodiments, the present inventions teaches a composition for use in urinals, comprising 2-ethylhexanol, dioctyl maleate, one or more aliphatic hydrocarbons comprising 13 to 23 carbon atoms, vegetable oil and triclosan.

In a fourth set of embodiments, the present invention teaches a urinal comprising a container holding collected urine and a drain that drains the container as said container is filled with urine, wherein the container further holds the formulation of claim 1.

In a fifth set of embodiments, the present invention teaches a method of suppressing odors in a urinal comprising adding a composition to a container for holding urine, the composition comprising: (a) one or more aliphatic alcohols, the aliphatic alcohols comprising 11 carbon atoms or less; (b) one or more esters selected from molecules of formula R¹—C(O)—O—R² and triglycerides of formula CH₂COOR⁶—CHCOOR⁷—CH₂—COOR⁸, R¹ comprising 6 to 24 carbon atoms, R² comprising 1 to 8 carbon atoms, R⁶ comprising 1 to 24 carbon atoms, R⁷ comprising 1 to 24 carbon atoms and R⁸ comprising 1 to 24 carbon atoms; (c) one or more molecules of formula R³—(O)—C(O)—R⁴—C(O)—(O)—R⁵, wherein R³, R⁴ and R⁵ are independently chosen from an alkyl or alkenyl moiety with 1 to 15 carbon atoms; (d) one or more aliphatic hydrocarbons, the aliphatic hydrocarbons comprising 6 to 23 carbon atoms; (e) optionally one or more vegetable oils; (f) optionally one or more unsaturated hydrocarbons, the unsaturated hydrocarbons comprising 6 to 24 carbon atoms; and (g) optionally one or more alicyclic hydrocarbons, the alyclic hydrocarbon comprising 6 to 23 carbon atoms.

These and other features of the invention are set forth herein.

### DESCRIPTION OF VARIOUS EMBODIMENTS

The present invention teaches waterless urinal liquid sealant formulations with a viscosity allowing sufficient urine permeability while maintaining low permeability to gaseous and volatile components. The formulations of the invention are also biodegradable and characterized by a freezing point that allows urine to pass through at temperatures below the water freezing point.

The formulations of the invention comprise alcohols comprising 11 carbon atoms or less, vegetable oils and/or their lower alkyl esters, fatty acid esters, esters of diacids, mineral oils and optionally alkyl alkoxylates of alcohols and amines. The alcohols preferably comprise 5 to 10 carbon atoms, and most preferably 7 to 9 carbon atoms. Particularly preferred are 2-alkyl-alkanols, such as 2-propyl-octanol, 2-ethyl-octanol, 2-methyl-octanol, 2-propyl-heptanol, 2-ethyl-heptanol, 2-methyl-heptanol, 2-propyl-hexanol, 2-ethyl-hexanol, 2-methyl-hexanol, 2-propyl-pentanol, 2-ethyl-pentanol and 2-methyl-pentanol. Most preferred are isomers of nonyl alcohol and isomers of octyl alcohol such as 2-ethylhexanol, isooctanol and n-octanol.

Preferred vegetable oils comprise, for example, canola oil, corn oil, linseed oil, rapeseed oil, cottonseed oil, castor oil, grape seed oil, safflower oil, peanut oil, rice bran oil, palm oil, coconut oil, soybean oil and sunflower oil. Preferred fatty acid esters are low viscosity oils, in particular fatty acids esters of formula R¹—C(O)—O—R² wherein R¹ comprises 6 to 24 carbon atoms and R² 1 to 8 carbon atoms. Particularly preferred are the esters found in “biodiesel”, a formulation manufactured by transesterification of the fatty acids of vegetable oils. The esters of biodiesel preferably comprise R² moieties with 1 to 5 carbon atoms, and most preferably comprise methyl esters or fatty acids. Fatty acid triglycerides of formula CH₂COOR⁶—CHCOOR⁷—CH₂—COOR⁸ such as those found in vegetable oil, canola oil, corn oil, soybean oil, sunflower oil and similar low viscosity oils, are also examples of fatty acid esters that can also be included in the formulations of the invention. Each triglyceride can independently comprise one or more types of unsaturated fatty acid chains, for instance oleic and linoleic acid, and saturated fatty acid chains, such as palmitic acid. More generally, each of R⁶, R⁷ and R⁸ is independently selected from saturated and unsaturated fatty acid chains comprising from 1 to 24 carbon atoms.

Preferred esters of diacids are of general formula R³—(O)—C(O)—R⁴—C(O)—(O)—R⁵, wherein R³, R⁴ and R⁵ are independently chosen from an alkyl or alkenyl moiety with 1 to 10 carbon atoms. Particularly preferred esters of diacids are maleates, succinates, alkylsuccinates and alkenylsuccinates, such as dioctyl maleate and dioctyl succinate. Without being bound to any particular theory, it appears that such esters of diacids contribute to the attainment of the desired viscosity and low freezing points of the compositions of the invention.

Preferred mineral oils can be saturated aliphatic hydrocarbons of formula C_{X}H_{2X+2}, unsaturated aliphatic hydrocarbons, alicyclic hydrocarbons of formula C_{X}H_{2X}, and mixtures thereof. Particularly preferred are mixtures of aliphatic hydrocarbons comprising 13 to 23 carbon atoms. Preferred commercially available mineral oils include AVATECH™ WHITE MINERAL OIL 40 (Avatar, University Park, Ill.), AVATECH™ WHITE MINERAL OIL 55 (Avatar, University Park, Ill.) and AVATECH™ WHITE MINERAL OIL 75 (Avatar, University Park, Ill.). Without being bound to any particular theory, it appears that, inter alia, such mineral oils also contribute to the attainment of the desired viscosity and low freezing points.

Preferred alkyl alkoxylates of alcohols and amines include ethoxylates and propoxylates, for example n-butyl polypropoxylates such as the commercially available PROBUTYL 14® (Croda Inc., Edison, N.J.).

One or more biocides may be added to the formulations of the invention. Lipophilic biocides are preferred, as the lipophilicity minimizes the leaching away of the biocide from the formulations of the invention into aqueous fluids. Preferred biocides are benzimidazoles, benzisothiazolinones, isothiazolinones, quaternary ammonium chlorides and bromides, hydantoin derivatives and aryl phenols. Particularly preferred are triclosan, a biocide commercially available as IRGASAN® 300 DP (Ciba Specialty Chemicals, Tarrytown, N.Y.) and halogenated aromatic compounds such as 4-chloro-3-methylphenol. Fragrances, for example lime or cherry fragrance, and dyes may also be included.

The following are specific examples of compositions of the present invention where the ingredients are mixed together in the percentages set forth in the examples.

### EXAMPLE 1

| | |
|---|---|
| 18.85% | 2-Ethylhexanol |
| 5.00% | Dioctyl Maleate |
| 5.00% | Premix (Premix of Dioctyl Maleate and dyes) |
| 30.00% | PROBUTYL ® 14 |
| 10.00% | Mineral Oil 40 |
| 30.00% | Soybean Oil |
| 00.15% | IRGASAN ® 300 DP |
| 1.00% | Fragrance, Cherry |

### EXAMPLE 2

| | |
|---|---|
| 20.00% | 2-Ethylhexanol |
| 14.85% | Dioctyl Maleate |
| 5.00% | Premix (Premix of Dioctyl Maleate and dyes) |
| 30.00% | Mineral Oil 40 |
| 30.00% | Vegetable Oil |
| 00.15% | IRGASAN ® 300 DP |

### EXAMPLE 3

| | |
|---|---|
| 15.6% | 2-Ethylhexanol |
| 20.8% | SoyGold ® 1000 (Methyl ester of Soybean oil) |
| | (Ag. Environmental Products, Omaha, NE) |
| 41.7% | Octyl Stearate |
| 4.17% | Premix of Dye in Dioctyl Maleate |
| 16.7% | Wesson Vegetable Oil |
| 0.2% | Irgasan ® DP 300R |
| 0.8% | Cherry Fragrance |

### EXAMPLE 4

| | |
|---|---|
| 18.75% | 2-Ethyihexanol |
| 25% | SoyGold ® 1000 |
| 50% | Probutyl ® 14 |
| 5% | Premix of Dye in Dioctyl Maleate |
| 0.25% | Irgasan DP 300R |
| 1% | Cherry Fragrance |

### EXAMPLE 5

| | |
|---|---|
| 15.6% | 2-Ethylhexanol |
| 20.8% | SoyGold ® 1000 |
| 16.7% | Wesson ® vegetable oil (ConAgra Foods, Omaha, NE) |
| 41.7% | Probutyl 14 |
| 4.17% | Premix of Dye in Dioctyl Maleate |
| 0.2% | Irgasan ® DP 300R |
| 0.83% | Cherry Fragrance |

### EXAMPLE 6

| | |
|---|---|
| 39.0% | 2-Ethylhexanol |
| 39.85% | Alfol ® 8 (Sasol, Houston, TX) |
| 15% | Dioctyl Maleate |
| 5% | Premix of Dioctyl Maleate with a dye |
| 1% | Fragrance |
| 0.15% | Irgasan 200 DP 300R |

### EXAMPLE 7

| | |
|---|---|
| 39.85% | Wesson ® Vegetable Oil |
| 39.0% | 2-Ethylhexanol |
| 15.0% | Dioctyl Maleate |
| 1% | Fragrance |
| 5% | Premix of Dioctyl Maleate with a dye |
| 0.15% | Irgasan ® DP 300 R |

The compositions of Examples 1 to 7 are lighter than water and so form a seal over a water solution simulating urine. Freezing point of the formulas is less than −10° C.

Although I have described my invention by reference to particular illustrative embodiments thereof, many changes and modifications of the invention may become apparent to those skilled in the art without departing from the spirit and scope of the invention. I therefore intend to include within the patent warranted hereon all such changes and modifications as may reasonably and properly be included within the scope of my contribution to the art.

1. A composition for use in urinals, comprising:
(a) one or more aliphatic alcohols, the aliphatic alcohols comprising 11 carbon atoms or less;
(b) one or more esters selected from molecules of formula R¹—C(O)—O—R² and triglycerides of formula CH₂COOR⁶—CHCOOR⁷—CH₂—COOR⁸, R¹ comprising 6 to 24 carbon atoms, R² comprising 1 to 8 carbon atoms, R⁶ comprising 1 to 24 carbon atoms, R⁷ comprising 1 to 24 carbon atoms and R⁸ comprising 1 to 24 carbon atoms;
(c) one or more molecules of formula R³—(O)—C(O)—R⁴—C(O)—(O)—R⁵, wherein R³, R⁴ and R⁵ are independently chosen from an alkyl or alkenyl moiety with 1 to 15 carbon atoms;
(d) one or more aliphatic hydrocarbons, the aliphatic hydrocarbons comprising 6 to 23 carbon atoms;
(e) optionally one or more vegetable oils;
(f) optionally one or more unsaturated hydrocarbons, the unsaturated hydrocarbons comprising 6 to 24 carbon atoms; and
(g) optionally one or more alicyclic hydrocarbons, the alyclic hydrocarbon comprising 6 to 23 carbon atoms.

2. The composition of claim 1, wherein the one or more aliphatic alcohols are selected from 2-propyl-octanol, 2-ethyl-octanol, 2-methyl-octanol, 2-propyl-heptanol, 2-ethyl-heptanol, 2-methyl-heptanol, 2-propyl-hexanol, 2-ethyl-hexanol, 2-methyl-hexanol, 2-propyl-pentanol, 2-ethyl-pentanol, 2-methyl-pentanol and any combination thereof.

3. The composition of claim 1, wherein the one or more aliphatic alcohols are selected from nonyl alcohol, 2-ethylhexanol, isooctanol, n-octanol and any combination thereof.

4. The composition of claim 1, wherein said triglycerides are triglycerides of saturated or unsaturated fatty acids.

5. The composition of claim 1, wherein said R² moiety is a —CH₃ group.

6. The composition of claim 1, wherein said molecules of formula R³—(O)—C(O)—R⁴—C(O)—(O)—R⁵ are selected from dioctyl maleate and dioctyl succinate or mixtures thereof.

7. The composition of claim 1, wherein said vegetable oils are selected from canola oil, corn oil, linseed oil, rapeseed oil, cottonseed oil, castor oil, grape seed oil, safflower oil, peanut oil, rice bran oil, palm oil, coconut oil, soybean oil, sunflower oil and mixtures thereof.

8. The composition of claim 1, further comprising one or more biocides.

9. The composition of claim 8, wherein at least one of the biocides is selected from triclosan, halogenated aromatic compounds or mixtures thereof.

10. A composition for use in urinals, comprising 2-ethylhexanol, dioctyl maleate, n-butyl-propoxylate, one or more aliphatic hydrocarbons comprising 13 to 23 carbon atoms, soybean oil and triclosan.

11. A composition for use in urinals, comprising 2-ethylhexanol, dioctyl maleate, one or more aliphatic hydrocarbons comprising 13 to 23 carbon atoms, vegetable oil and triclosan.

12. A urinal comprising a container holding collected urine and a drain that drains the container as said container is filled with urine, wherein the container further holds the formulation of claim 1.

13. A method of suppressing odors in a urinal comprising adding a composition to a container for holding urine, the composition comprising:
(a) one or more aliphatic alcohols, the aliphatic alcohols comprising 11 carbon atoms or less;
(b) one or more esters selected from molecules of formula R¹—C(O)—O—R² and triglycerides of formula CH₂COOR⁶—CHCOOR⁷—CH₂—COOR⁸, R¹ comprising 6 to 24 carbon atoms, R² comprising 1 to 8 carbon atoms, R⁶ comprising 1 to 24 carbon atoms, R⁷ comprising 1 to 24 carbon atoms and R⁸ comprising 1 to 24 carbon atoms;
(c) one or more molecules of formula R³—(O)—C(O)—R⁴—C(O)—(O)—R⁵, wherein R³, R⁴ and R⁵ are independently chosen from an alkyl or alkenyl moiety with 1 to 15 carbon atoms;
(d) one or more aliphatic hydrocarbons, the aliphatic hydrocarbons comprising 6 to 23 carbon atoms;
(e) optionally one or more vegetable oils;
(f) optionally one or more unsaturated hydrocarbons, the unsaturated hydrocarbons comprising 6 to 24 carbon atoms; and
(g) optionally one or more alicyclic hydrocarbons, the alyclic hydrocarbon comprising 6 to 23 carbon atoms.

14. The method of claim 13, wherein the one or more aliphatic alcohols are selected from 2-propyl-octanol, 2-ethyl-octanol, 2-methyl-octanol, 2-propyl-heptanol, 2-ethyl-heptanol, 2-methyl-heptanol, 2-propyl-hexanol, 2-ethyl-hexanol, 2-methyl-hexanol, 2-propyl-pentanol, 2-ethyl-pentanol, 2-methyl-pentanol and any combination thereof.

15. The method of claim 14, wherein the one or more aliphatic alcohols are selected from nonyl alcohol, 2-ethylhexanol, isooctanol, n-octanol and any combination thereof.

16. The method of claim 1, wherein said R² is a —CH₃ group.

17. The method of claim 13, wherein the triglycerides are triglycerides comprising saturated and optionally unsaturated fatty acid moieties.

18. The method of claim 13, wherein the molecules of formula R³—(O)—C(O)—R⁴—C(O)—(O)—R⁵ are selected from dioctyl maleate and dioctyl succinate or mixtures thereof.

19. The method of claim 13, wherein said vegetable oils are selected from canola oil, corn oil, linseed oil, rapeseed oil, cottonseed oil, castor oil, grape seed oil, safflower oil, peanut oil, rice bran oil, palm oil, coconut oil, soybean oil, sunflower oil and mixtures thereof.

20. The method of claim 13, wherein the composition further comprises one or more biocides.

21. The method of claim 13, wherein at least one of the biocides is selected from triclosan, halogenated aromatic compounds or mixtures thereof.

22. The method of claim 13, wherein the composition comprises 2-ethylhexanol, dioctyl maleate, n-butyl-propoxylate, one or more aliphatic hydrocarbons comprising 13 to 23 carbon atoms, soybean oil and triclosan.

23. The method of claim 13, wherein the composition comprises 2-ethylhexanol, dioctyl maleate, one or more aliphatic hydrocarbons comprising 13 to 23 carbon atoms, vegetable oil and triclosan.
